(19) United States
(12) Patent Application Publication
(10) Pub. No.: US 2016/0361970 A1
(43) Pub. Date: Dec. 15, 2016

(54) CONTROLLING VEHICLE FUNCTIONS BASED ON INPUT FROM A PORTABLE CONSUMER ELECTRONICS DEVICE

(71) Applicant: GM Global Technology Operations LLC, Detroit, MI (US)

(72) Inventors: Paul H. PEBBLES, Novi, MI (US); Samer ZAKHEM, Troy, MI (US); Cem SARAYDAR, Birmingham, MI (US)

(21) Appl. No.: 14/734,353

(22) Filed: Jun. 9, 2015

(51) Int. Cl. B60H 1/00; A61B 5/18; B60W 30/08

(52) U.S. Cl. CPC B60H 1/00742; B60W 30/08; A61B 5/18; B60W 2540/26; B60W 2900/00; B60W 2710/20; B60W 2710/18; B60W 2400/00

(57) ABSTRACT

A system and method of controlling a vehicle function at a vehicle using data received from a portable consumer electronic device includes: establishing a communication link between the vehicle and the portable consumer electronic device; receiving data indicative of a human condition at the vehicle from the portable consumer electronic device via the communication link; generating a command that controls one or more portions of vehicle electronics based on the data indicative of the human condition; and transmitting the command over a vehicle bus.

### TECHNICAL FIELD

The present invention relates to vehicle operation and, more particularly, to controlling vehicle functions based on input from a portable consumer electronics device.

### BACKGROUND

Vehicles are frequently equipped with a wide variety of vehicle electronics that monitor and control vehicle functions. Vehicles include commonly-known inputs used to control the vehicle, such as a steering wheel, an accelerator pedal, and a brake pedal. But these inputs or vehicle functions can be monitored and control of the vehicle altered based on data gathered at one or more vehicle sensors that is processed by the vehicle electronics.

An example of this can be explained using a lane departure system available on some modern vehicles. A vehicle can use a sensor to determine when a vehicle moves outside of a lane on a road. The sensor can be implemented as a camera that monitors the lane boundaries. When the vehicle leaves a lane, the vehicle can respond by shaking the steering wheel and/or controlling the steering wheel to guide the vehicle back into the lane. But such a system is reactive to physical actions made by the driver. The vehicle alters control of the vehicle only after it detects a deviation from normal based on those physical actions (i.e., driving outside of a lane). It would be helpful to gather data that may not necessarily rely on physical actions from the driver and do so using a source that is separate from the vehicle electronics.

### SUMMARY

According to an embodiment of the invention, there is provided a method of controlling a vehicle function at a vehicle using data received from a portable consumer electronic device. The method includes establishing a communication link between the vehicle and the portable consumer electronic device; receiving data indicative of a human condition at the vehicle from the portable consumer electronic device via the communication link; generating a command that controls one or more portions of vehicle electronics based on the data indicative of the human condition; and transmitting the command over a vehicle bus.

According to another embodiment of the invention, there is provided a method of controlling a vehicle function at a vehicle using data received from a portable consumer electronic device. The method includes establishing a communication link between the vehicle and the portable consumer electronic device; receiving data indicative of a human condition at the vehicle from the portable consumer electronic device via the communication link; converting the received data from a format used by the portable consumer electronic device to a format used by a vehicle bus; generating a command that controls one or more portions of vehicle electronics using the received data; and transmitting the command over the vehicle bus.

According to yet another embodiment of the invention, there is provided a method of controlling a vehicle function at a vehicle using data received from a portable consumer electronic device. The method includes storing at the vehicle a plurality of thresholds associated with a human condition; storing at the vehicle one or more changes in vehicle function for each threshold; establishing a communication link between the vehicle and the portable consumer electronic device; receiving data indicative of the human condition at the vehicle from the portable consumer electronic device via the communication link; comparing the received data with the plurality of thresholds at the vehicle; determining which of the plurality of thresholds is met; and commanding one or more elements of vehicle electronics to carry out the changes in vehicle function(s) stored with the threshold determined to have been met.

### BRIEF DESCRIPTION OF THE DRAWINGS

One or more embodiments of the invention will hereinafter be described in conjunction with the appended drawings, wherein like designations denote like elements, and wherein:

FIG. 1 is a block diagram depicting an embodiment of a communications system that is capable of utilizing the method disclosed herein; and

FIG. 2 is a flow chart depicting an implementation of a method of controlling a vehicle function at a vehicle using data received from a portable consumer electronic device.

### DETAILED DESCRIPTION OF THE ILLUSTRATED EMBODIMENTS

The system and method described below gathers data about the condition of a vehicle occupant using a portable consumer electronics device (PCED) and communicates that data to a vehicle where it can alter the operation of one or more vehicle systems. Vehicle occupants often carry PCEDs that can gather data reflecting the condition of a vehicle occupant, such as individual health measurements or a compilation of health measurements that indicate overall human awareness or drowsiness. The PCEDs can include “wearable technology” such as quantified self devices as well as smart phones. The data can be transmitted to the vehicle and then be used to alter vehicle functions or operational settings used by one or more portions of the vehicle electronics.

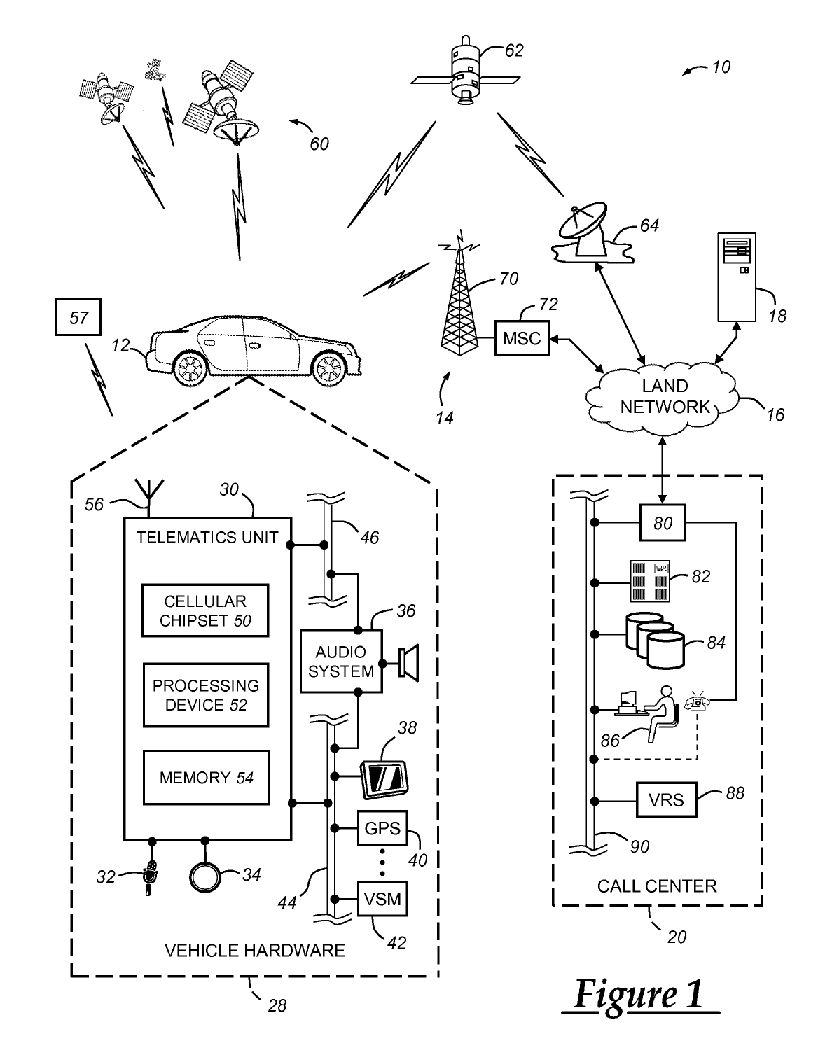

With reference to FIG. 1, there is shown an operating environment that comprises a mobile vehicle communications system 10 and that can be used to implement the method disclosed herein. Communications system 10 generally includes a vehicle 12, one or more wireless carrier systems 14, a land communications network 16, a computer 18, and a call center 20. It should be understood that the disclosed method can be used with any number of different systems and is not specifically limited to the operating environment shown here. Also, the architecture, construction, setup, and operation of the system 10 and its individual components are generally known in the art. Thus, the following paragraphs simply provide a brief overview of one such communications system 10; however, other systems not shown here could employ the disclosed method as well.

Vehicle 12 is depicted in the illustrated embodiment as a passenger car, but it should be appreciated that any other vehicle including motorcycles, trucks, sports utility vehicles (SUVs), recreational vehicles (RVs), marine vessels, aircraft, etc., can also be used. Some of the vehicle electronics 28 is shown generally in FIG. 1 and includes a telematics unit 30, a microphone 32, one or more pushbuttons or other control inputs 34, an audio system 36, a visual display 38, and a GPS module 40 as well as a number of vehicle system modules (VSMs) 42. Some of these devices can be connected directly to the telematics unit such as, for example, the microphone 32 and pushbutton(s) 34, whereas others are indirectly connected using one or more network connections, such as a communications bus 44 or an entertainment bus 46. Examples of suitable network connections include a controller area network (CAN), a media oriented system transfer (MOST), a local interconnection network (LIN), a local area network (LAN), and other appropriate connections such as Ethernet or others that conform with known ISO, SAE and IEEE standards and specifications, to name but a few.

Telematics unit 30 can be an OEM-installed (embedded) or aftermarket device that is installed in the vehicle and that enables wireless voice and/or data communication over wireless carrier system 14 and via wireless networking. This enables the vehicle to communicate with call center 20, other telematics-enabled vehicles, or some other entity or device. The telematics unit preferably uses radio transmissions to establish a communications channel (a voice channel and/or a data channel) with wireless carrier system 14 so that voice and/or data transmissions can be sent and received over the channel. By providing both voice and data communication, telematics unit 30 enables the vehicle to offer a number of different services including those related to navigation, telephony, emergency assistance, diagnostics, infotainment, etc. Data can be sent either via a data connection, such as via packet data transmission over a data channel, or via a voice channel using techniques known in the art. For combined services that involve both voice communication (e.g., with a live advisor or voice response unit at the call center 20) and data communication (e.g., to provide GPS location data or vehicle diagnostic data to the call center 20), the system can utilize a single call over a voice channel and switch as needed between voice and data transmission over the voice channel, and this can be done using techniques known to those skilled in the art.

According to one embodiment, telematics unit 30 utilizes cellular communication according to either GSM, CDMA, or LTE standards and thus includes a standard cellular chipset 50 for voice communications like hands-free calling, a wireless modem for data transmission, an electronic processing device 52, one or more digital memory devices 54, and a dual antenna 56. It should be appreciated that the modem can either be implemented through software that is stored in the telematics unit and is executed by processor 52, or it can be a separate hardware component located internal or external to telematics unit 30. The modem can operate using any number of different standards or protocols such as LTE, EVDO, CDMA, GPRS, and EDGE. Wireless networking between the vehicle and other networked devices can also be carried out using telematics unit 30. For this purpose, telematics unit 30 can be configured to communicate wirelessly according to one or more wireless protocols, including short range wireless communication (SRWC) such as any of the IEEE 802.11 protocols, WiMAX, ZigBee™, Wi-Fi direct, Bluetooth, Bluetooth Low Energy (LE), or near field communication (NFC). When used for packet-switched data communication such as TCP/IP, the telematics unit can be configured with a static IP address or can set up to automatically receive an assigned IP address from another device on the network such as a router or from a network address server.

One of the networked devices that can communicate with the telematics unit 30 is a PCED 57. As pointed out above, the PCED 57 can be implemented using a quantified self device or smart phone that is carried by or placed in close proximity of the vehicle occupant. The PCED 57 can measure a wide array of human activity or biometrics reflecting human health or condition using electronic hardware that can include any combination of a heart rate sensor, a temperature sensor, a three-dimensional accelerometer, a clock, a short-range wireless transceiver, or a photodiode. The electronic hardware can measure human movement, temperature, heart rate, and/or blood pressure of a vehicle occupant and translate the measurements into data. The PCED 57 implemented as a quantified self device may also be referred to as an “activity monitor,” a “wearable device” or “wearable computing.” Existing quantifiable self devices include the Apple™ Watch and the Fitbit™.

The PCED 57 can also be implemented using a smart phone that includes computer processing capability, a transceiver capable of communicating using a short-range wireless protocol, and a visual display. Any one or more of the electronic hardware identified above with respect to the quantified self device could also be used in the smart phone. In some implementations, the visual display also includes a touch-screen graphical user interface and/or a GPS module capable of receiving GPS satellite signals and generating GPS coordinates based on those signals. Examples of the smart phone include the iPhone™ manufactured by Apple, Inc. and the Droid™ manufactured by Motorola, Inc. as well as others. While the smart phone may include the ability to communicate via cellular communications using the wireless carrier system 14, this is not always the case. For instance, Apple manufactures devices such as the various models of the iPad™ and iPod Touch™ that include the processing capability, the visual display, and the ability to communicate over a short-range wireless communication link. However, the iPod Touch™ and some iPads™ do not have cellular communication capabilities. Even so, these and other similar devices may be used or considered a type of PCED, such as the smart phone, for the purposes of the method described herein.

Processor 52 can be any type of device capable of processing electronic instructions including microprocessors, microcontrollers, host processors, controllers, vehicle communication processors, and application specific integrated circuits (ASICs). It can be a dedicated processor used only for telematics unit 30 or can be shared with other vehicle systems. Processor 52 executes various types of digitally-stored instructions, such as software or firmware programs stored in memory 54, which enable the telematics unit to provide a wide variety of services. For instance, processor 52 can execute programs or process data to carry out at least a part of the method discussed herein.

Telematics unit 30 can be used to provide a diverse range of vehicle services that involve wireless communication to and/or from the vehicle. Such services include: turn-by-turn directions and other navigation-related services that are provided in conjunction with the GPS-based vehicle navigation module 40; airbag deployment notification and other emergency or roadside assistance-related services that are provided in connection with one or more collision sensor interface modules such as a body control module (not shown); diagnostic reporting using one or more diagnostic modules; and infotainment-related services where music, webpages, movies, television programs, videogames and/or other information is downloaded by an infotainment module (not shown) and is stored for current or later playback. The above-listed services are by no means an exhaustive list of all of the capabilities of telematics unit 30, but are simply an enumeration of some of the services that the telematics unit is capable of offering. Furthermore, it should be understood that at least some of the aforementioned modules could be implemented in the form of software instructions saved internal or external to telematics unit 30, they could be hardware components located internal or external to telematics unit 30, or they could be integrated and/or shared with each other or with other systems located throughout the vehicle, to cite but a few possibilities. In the event that the modules are implemented as VSMs 42 located external to telematics unit 30, they could utilize vehicle bus 44 to exchange data and commands with the telematics unit.

GPS module 40 receives radio signals from a constellation 60 of GPS satellites.

From these signals, the module 40 can determine vehicle position that is used for providing navigation and other position-related services to the vehicle driver. Navigation information can be presented on the display 38 (or other display within the vehicle) or can be presented verbally such as is done when supplying turn-by-turn navigation. The navigation services can be provided using a dedicated in-vehicle navigation module (which can be part of GPS module 40), or some or all navigation services can be done via telematics unit 30, wherein the position information is sent to a remote location for purposes of providing the vehicle with navigation maps, map annotations (points of interest, restaurants, etc.), route calculations, and the like. The position information can be supplied to call center 20 or other remote computer system, such as computer 18, for other purposes, such as fleet management. Also, new or updated map data can be downloaded to the GPS module 40 from the call center 20 via the telematics unit 30.

Apart from the audio system 36 and GPS module 40, the vehicle 12 can include other vehicle system modules (VSMs) 42 in the form of electronic hardware components that are located throughout the vehicle and typically receive input from one or more sensors and use the sensed input to perform diagnostic, monitoring, control, reporting and/or other functions. Each of the VSMs 42 is preferably connected by communications bus 44 to the other VSMs, as well as to the telematics unit 30, and can be programmed to run vehicle system and subsystem diagnostic tests. As examples, one VSM 42 can be an engine control module (ECM) that controls various aspects of engine operation such as fuel ignition and ignition timing, another VSM 42 can be a powertrain control module that regulates operation of one or more components of the vehicle powertrain, and another VSM 42 can be a body control module that governs various electrical components located throughout the vehicle, like the vehicle's power door locks and headlights. According to one embodiment, the engine control module is equipped with on-board diagnostic (OBD) features that provide myriad real-time data, such as that received from various sensors including vehicle emissions sensors, and provide a standardized series of diagnostic trouble codes (DTCs) that allow a technician to rapidly identify and remedy malfunctions within the vehicle.

A VSM 42 can be used to carry out collision avoidance systems in some implementations. Collision avoidance systems use vehicle sensors to alert the driver to a possible crash and, in some cases, exert control over vehicle inputs (e.g., steering wheel and/or brake pedal) to avoid the crash. The VSM 42 can receive data from sensors in the form of a camera and a radar or laser to determine position of the vehicle 12 in a lane and the proximity of the vehicle 12 to other objects. In one implementation, the collision avoidance system can exert moderate braking when the vehicle 12 is within a first range of a detected object and heavy braking when the vehicle is within a second, closer range of the detected object. In another possible implementation, the collision avoidance system can determine when the vehicle 12 passes a first proximity boundary of a lane and issue a audible or visual warning. When the vehicle 12 moves past the first boundary and beyond a second proximity boundary, the collision avoidance system can adjust the steering wheel of the vehicle 12 to keep the vehicle 12 within the lane. As is appreciated by those skilled in the art, the above-mentioned VSMs are only examples of some of the modules that may be used in vehicle 12, as numerous others are also possible.

Vehicle electronics 28 also includes a number of vehicle user interfaces that provide vehicle occupants with a means of providing and/or receiving information, including microphone 32, pushbuttons(s) 34, audio system 36, and visual display 38. As used herein, the term ‘vehicle user interface’ broadly includes any suitable form of electronic device, including both hardware and software components, which is located on the vehicle and enables a vehicle user to communicate with or through a component of the vehicle. Microphone 32 provides audio input to the telematics unit to enable the driver or other occupant to provide voice commands and carry out hands-free calling via the wireless carrier system 14. For this purpose, it can be connected to an on-board automated voice processing unit utilizing human-machine interface (HMI) technology known in the art. The pushbutton(s) 34 allow manual user input into the telematics unit 30 to initiate wireless telephone calls and provide other data, response, or control input. Separate pushbuttons can be used for initiating emergency calls versus regular service assistance calls to the call center 20. Audio system 36 provides audio output to a vehicle occupant and can be a dedicated, stand-alone system or part of the primary vehicle audio system. In some implementations, the audio system 36 can include its own antenna apart from the dual antenna 56 for communicating with other electronics devices via short-range wireless protocols, such as Wi-Fi or Bluetooth LE. According to the particular embodiment shown here, audio system 36 is operatively coupled to both vehicle bus 44 and entertainment bus 46 and can provide AM, FM and satellite radio, CD, DVD and other multimedia functionality. This functionality can be provided in conjunction with or independent of the infotainment module described above. Visual display 38 is preferably a graphics display, such as a touch screen on the instrument panel or a heads-up display reflected off of the windshield, and can be used to provide a multitude of input and output functions. Various other vehicle user interfaces can also be utilized, as the interfaces of FIG. 1 are only an example of one particular implementation.

Wireless carrier system 14 is preferably a cellular telephone system that includes a plurality of cell towers 70 (only one shown), one or more mobile switching centers (MSCs) 72, as well as any other networking components required to connect wireless carrier system 14 with land network 16. Each cell tower 70 includes sending and receiving antennas and a base station, with the base stations from different cell towers being connected to the MSC 72 either directly or via intermediary equipment such as a base station controller. Cellular system 14 can implement any suitable communications technology, including for example, analog technologies such as AMPS, or the newer digital technologies such as CDMA (e.g., CDMA2000) or GSM/GPRS. As will be appreciated by those skilled in the art, various cell tower/base station/MSC arrangements are possible and could be used with wireless system 14. For instance, the base station and cell tower could be co-located at the same site or they could be remotely located from one another, each base station could be responsible for a single cell tower or a single base station could service various cell towers, and various base stations could be coupled to a single MSC, to name but a few of the possible arrangements.

Apart from using wireless carrier system 14, a different wireless carrier system in the form of satellite communication can be used to provide uni-directional or bi-directional communication with the vehicle. This can be done using one or more communication satellites 62 and an uplink transmitting station 64. Uni-directional communication can be, for example, satellite radio services, wherein programming content (news, music, etc.) is received by transmitting station 64, packaged for upload, and then sent to the satellite 62, which broadcasts the programming to subscribers. Bi-directional communication can be, for example, satellite telephony services using satellite 62 to relay telephone communications between the vehicle 12 and station 64. If used, this satellite telephony can be utilized either in addition to or in lieu of wireless carrier system 14.

Land network 16 may be a conventional land-based telecommunications network that is connected to one or more landline telephones and connects wireless carrier system 14 to call center 20. For example, land network 16 may include a public switched telephone network (PSTN) such as that used to provide hardwired telephony, packet-switched data communications, and the Internet infrastructure. One or more segments of land network 16 could be implemented through the use of a standard wired network, a fiber or other optical network, a cable network, power lines, other wireless networks such as wireless local area networks (WLANs), or networks providing broadband wireless access (BWA), or any combination thereof. Furthermore, call center 20 need not be connected via land network 16, but could include wireless telephony equipment so that it can communicate directly with a wireless network, such as wireless carrier system 14.

Computer 18 can be one of a number of computers accessible via a private or public network such as the Internet. Each such computer 18 can be used for one or more purposes, such as a web server accessible by the vehicle via telematics unit 30 and wireless carrier 14. Other such accessible computers 18 can be, for example: a service center computer where diagnostic information and other vehicle data can be uploaded from the vehicle via the telematics unit 30; a client computer used by the vehicle owner or other subscriber for such purposes as accessing or receiving vehicle data or to setting up or configuring subscriber preferences or controlling vehicle functions; or a third party repository to or from which vehicle data or other information is provided, whether by communicating with the vehicle 12 or call center 20, or both. A computer 18 can also be used for providing Internet connectivity such as DNS services or as a network address server that uses DHCP or other suitable protocol to assign an IP address to the vehicle 12.

Call center 20 is designed to provide the vehicle electronics 28 with a number of different system back-end functions and, according to the exemplary embodiment shown here, generally includes one or more switches 80, servers 82, databases 84, live advisors 86, as well as an automated voice response system (VRS) 88, all of which are known in the art. These various call center components are preferably coupled to one another via a wired or wireless local area network 90. Switch 80, which can be a private branch exchange (PBX) switch, routes incoming signals so that voice transmissions are usually sent to either the live adviser 86 by regular phone or to the automated voice response system 88 using VoIP. The live advisor phone can also use VoIP as indicated by the broken line in FIG. 1. VoIP and other data communication through the switch 80 is implemented via a modem (not shown) connected between the switch 80 and network 90. Data transmissions are passed via the modem to server 82 and/or database 84. Database 84 can store account information such as subscriber authentication information, vehicle identifiers, profile records, behavioral patterns, and other pertinent subscriber information. Data transmissions may also be conducted by wireless systems, such as 802.1 lx, GPRS, and the like. Although the illustrated embodiment has been described as it would be used in conjunction with a manned call center 20 using live advisor 86, it will be appreciated that the call center can instead utilize VRS 88 as an automated advisor or, a combination of VRS 88 and the live advisor 86 can be used.

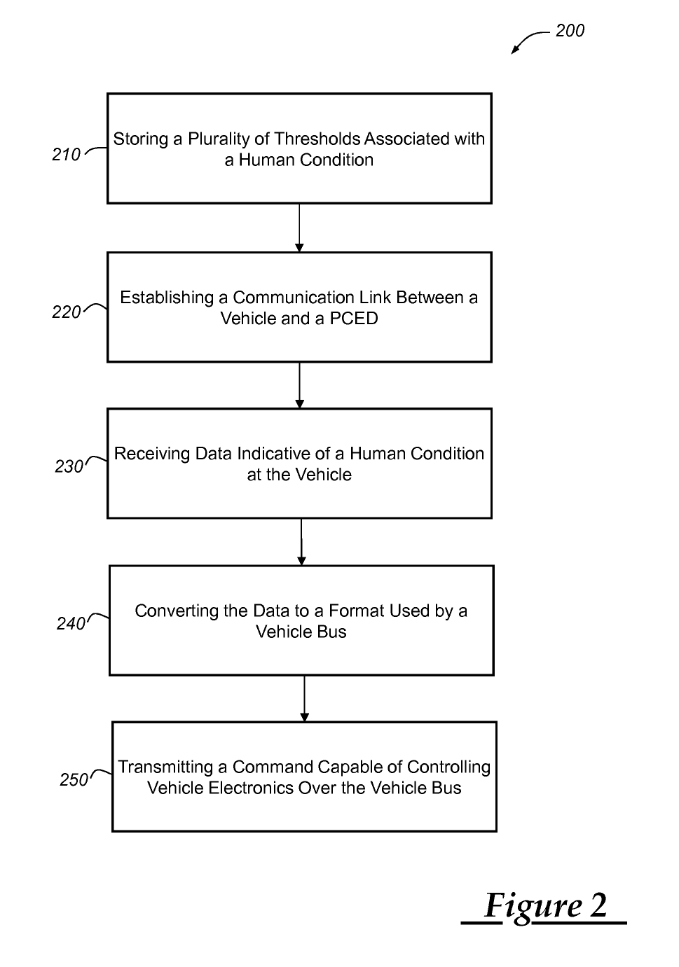

Turning now to FIG. 2, there is shown an implementation of a method 200 of controlling a vehicle function at the vehicle 12 using data received from the portable consumer electronic device (PCED) 57. The method 200 begins at step 210 by storing at the vehicle 12 a plurality of thresholds associated with a human condition (also referred to as human health) and one or more changes in vehicle function for each threshold. Examples of human conditions include human movement, temperature, heart rate, and/or blood pressure as noted above. Each of these human conditions can be quantified by selecting units used to express relative measurements. Movement can be quantified using any one of a number of distance measurements, while temperature can be measured using Celsius or Fahrenheit scales. Heart rates can be measured using beats-per-minute (BPM) and blood pressure using systolic over diastolic measurements. The method 200 will be described using the heart rate of the vehicle occupant as an exemplary measured human condition and changes to vehicle thresholds used by a vehicle collision avoidance system as an exemplary vehicle threshold and vehicle function. However, it should be appreciated that different human conditions, vehicle thresholds, and vehicle functions can be used instead of, or in addition to, the heart rate of a vehicle occupant and collision avoidance systems.

Depending on the vehicle function to be monitored and controlled based on the human conditions, one or more thresholds can be established for the vehicle function. As noted above, the collision avoidance system can use different thresholds for distance to an object or distance to a lane boundary and each of those thresholds can be changed in response to different measurements of a human condition. For example, the distance thresholds of the collision avoidance systems apply moderate and heavy braking, respectively, when the first and second vehicle function thresholds are reached. And these vehicle function thresholds can be altered based on a human condition threshold. Collision avoidance systems can establish its range values between vehicle 12 and object based on the human condition thresholds.

In one implementation, a plurality of logical values numbered zero through three can be associated with a range of heart beat measurements. Logical zero can be associated with a heart beat rate below 50 BPM. This human threshold can indicate that the vehicle occupant is in shock and the vehicle function thresholds for distance between the vehicle 12 and the lane boundaries can be increased such that the system intervenes sooner as the vehicle 12 approaches the lane boundary. Logical one can be associated with heart beat measurements between 50 and 100 BPM. The heart beat measurements of logical one can indicate that the vehicle occupant has a heart beat that is considered normal when at rest. The vehicle function thresholds of the collision avoidance system can be set to default values. Logical two can be associated with heart beat measurements over 100 BPM. Logical two heart beat measurements can indicate that the vehicle occupant is feeling stressed. The distance thresholds can then be increased such that intervention via moderate and heavy braking occurs sooner than it would during the logical one condition. And logical three can be associated with an abnormal measured heart beat. This threshold can indicate that the vehicle occupant is experiencing a health crisis, such as a heart attack, and cause the initiation of an emergency call to the call center 20 via the vehicle telematics unit 30. The method 200 proceeds to step 220.

At step 220, a communication link is established between the vehicle 12 and the PCED 57. The PCED 57 can be carried or worn by the vehicle occupant and, when within range of the short-range communication capabilities of the vehicle 12, the PCED 57 can link with the vehicle 12 using any one of a number of short-range wireless communication protocols, such as Bluetooth LE or Wi-Fi. Specifically, the PCED 57 and the vehicle telematics unit 30 can maintain and use the communication link. But in some configurations, the audio system 36 can use its own antenna (not shown) to support the communication link. In other implementations, the PCED 57 can be linked to the vehicle 12 using a data cable that is received by an OBD II data link connector (DLC). In either implementation, the vehicle 12 can receive packetized data that includes information regarding the human condition of the vehicle occupant sent from the PCED 57. The method 200 proceeds to step 230.

At step 230, data indicative of a human condition is received at the vehicle 12 from the PCED 57 via the communication link. When the vehicle occupant is inside or nearby the vehicle 12, the PCED 57 can continuously or periodically transmit the human condition data to the vehicle 12. With respect to the collision avoidance system, the PCED 57 may measure a heart rate over a period of time and periodically provide the measured heart rate to the vehicle 12. In one implementation, the PCED 57 may measure the vehicle occupant's heart rate over a period of 10 seconds and then extrapolate the measured heart beats over that period into BPM. The PCED 57 can then transmit the measured heart rate to the vehicle 12. In this sense, the vehicle 12 can receive an updated value from the PCED 57 every 10 seconds. The method 200 proceeds to step 240.

At step 240, the received data is converted from a format native to the PCED 57 to a format used by the communications bus 44. In some implementations, the data including information about the human condition of the vehicle occupant may not be compatible with the communications bus 44 on the vehicle 12. For example, the communications bus 44 may be implemented as a CAN bus that communicates data in a format that is not supported by the PCED 57. When this occurs, the vehicle telematics unit 30 or other portion of the vehicle electronics 28 can read the information provided by the PCED 57 and encode that information into parameter ID (PID) messages capable of being transported by the communications bus 44. This conversion can be handled by an application programming interface (API) that is stored at the vehicle 12. The human condition data can then be directed by the vehicle electronics 28 to an appropriate destination. In this example, the vehicle telematics unit 30 can receive the data and transmit it over the communication bus 44 to the VSM 42 controlling the collision avoidance system. The method 200 proceeds to step 250.

At step 250, a command that is capable of controlling one or more portions of vehicle electronics 28 using the received data is generated and transmitted over the vehicle bus 44. The vehicle electronics 28 can be controlled in a way that responds to the human condition data automatically to change vehicle operation without requiring any initiation or input from the vehicle occupant. For example, the vehicle 12 is configured to automatically adjust a collision warning distance based on measured human condition or biometrics. The VSM 42 operating the collision avoidance system can receive the human condition data (in this example, the measured heart beat) and then compare the measured heart beat of the vehicle occupant with the ranges of the logical values discussed above. The VSM 42 can identify a logical value that matches the measured heart beat and determine whether the existing thresholds used by the system should be changed or maintained. If the existing thresholds of the collision avoidance system differ from those associated with the logical value, the VSM 42 can change them so that they conform to what is specified by the matched logical value. The method 200 then ends.

It is to be understood that the foregoing is a description of one or more embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. For example, the foregoing method could be applied to the audio system or a heating, ventilation, and air conditioning (HVAC) system each of which are types of vehicle functions. Human conditions can be monitored and particular music can be selected based on heart rate or blood pressure measurements. Also, the HVAC system can be controlled based on a human condition, such as a measured temperature of the vehicle occupant. These and other embodiments, changes, and modifications are intended to come within the scope of the appended claims.

As used in this specification and claims, the terms “e.g.,” “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

1. A method of controlling a vehicle function at a vehicle using data received from a portable consumer electronic device, comprising the steps of:
(a) establishing a communication link between the vehicle and the portable consumer electronic device;
(b) receiving data indicative of a human condition at the vehicle from the portable consumer electronic device via the communication link;
(c) generating a command that controls one or more portions of vehicle electronics based on the data indicative of the human condition; and
(d) transmitting the command over a vehicle bus.

2. The method of claim 1, wherein the communication link is established via a short-range wireless communication protocol.

3. The method of claim 1, wherein the human condition further comprises a heart beat rate measurement or a blood pressure measurement.

4. The method of claim 1, wherein the vehicle electronics further comprise a collision avoidance system.

5. The method of claim 1, wherein the vehicle electronics further comprise an audio system or a heating, ventilation, and air conditioning (HVAC) system.

6. The method of claim 1, further comprising the step of changing one or more vehicle function thresholds based on the data indicative of the human condition.

7. The method of claim 1, further comprising the step of establishing a plurality of logical values each of which is associated with a range of human condition measurements.

8. The method of claim 1, further comprising the step of converting the received data from a protocol used by the portable consumer electronic device to another, different protocol used by the vehicle.

9. A method of controlling a vehicle function at a vehicle using data received from a portable consumer electronic device, comprising the steps of:
(a) establishing a communication link between the vehicle and the portable consumer electronic device;
(b) receiving data indicative of a human condition at the vehicle from the portable consumer electronic device via the communication link;
(c) converting the received data from a format used by the portable consumer electronic device to a format used by a vehicle bus;
(d) generating a command that controls one or more portions of vehicle electronics using the received data; and
(e) transmitting the command over the vehicle bus.

10. The method of claim 9, wherein the communication link is established via a short-range wireless communication protocol.

11. The method of claim 9, wherein the human condition further comprises a heart beat rate measurement or a blood pressure measurement.

12. The method of claim 9, wherein the vehicle electronics further comprise a collision avoidance system.

13. The method of claim 9, further comprising the step of changing one or more vehicle function thresholds based on the data indicative of the human condition.

14. The method of claim 9, further comprising the step of establishing a plurality of logical values each of which is associated with a range of human condition measurements.

15. A method of controlling a vehicle function at a vehicle using data received from a portable consumer electronic device, comprising the steps of:
(a) storing at the vehicle a plurality of thresholds associated with a human condition;
(b) storing at the vehicle one or more changes in vehicle function for each threshold;
(c) establishing a communication link between the vehicle and the portable consumer electronic device;
(d) receiving data indicative of the human condition at the vehicle from the portable consumer electronic device via the communication link;
(e) comparing the received data with the plurality of thresholds at the vehicle;
(f) determining which of the plurality of thresholds is met; and
(g) commanding one or more elements of vehicle electronics to carry out the change(s) in vehicle function stored with the threshold determined to have been met.

16. The method of claim 15, wherein the communication link is established via a short-range wireless communication protocol.

17. The method of claim 15, wherein the human condition further comprises a heart beat rate measurement or a blood pressure measurement.

18. The method of claim 15, wherein the vehicle electronics further comprise a collision avoidance system.

19. The method of claim 15, further comprising the step of establishing a plurality of logical values each of which is associated with a range of human condition measurements.
